SEQUENCE LISTING (2 sequences)

SEQ ID NO: 1 - protein, 18 aa, Artificial Sequence
  Synthetic Construct
TGAGGGGACTTTCCCAGG

SEQ ID NO: 2 - protein, 18 aa, Artificial Sequence
  Synthetic Construct
TGAGGCGACTTTCCCAGG

(19) United States
(12) Patent Application Publication
(10) Pub. No.: US 2011/0117121 A1
(43) Pub. Date: May 19, 2011

(54) COMPOSITIONS FOR TREATMENT AND INHIBITION OF PAIN

(76) Inventors: James Dao, Henderson, NV (US); Jeff Dao, San Mateo, CA (US); Leslie Wilson, Carpinteria, CA (US); William Gerwick, Corvallis, OR (US); Emin Oroudjev, Santa Barbara, CA (US)

(21) Appl. No.: 12/446,561

(22) PCT Filed: Oct. 26, 2007
(86) PCT No.: PCT/US07/22763

§ 371 Date: Apr. 21, 2009

(30) Foreign Application Priority Data
Oct. 27, 2006 (US) 60855024

(51) Int. Cl. A61K 36/06; A61P 25/00

(52) U.S. Cl. 424/195.15

(57) ABSTRACT

Methods and compositions for therapy of pain are provided. Compositions comprising therapeutically effective amounts of two or more of an extract of Ganoderma lucidum, an extract of Salvia miltiorrhiza and an extract of Scutellaria barbata and optionally a therapeutically effective amount of an extract of Hippophae rhamnoides are provided. Novel synergistic effects of the use of these compounds in combination therapy are disclosed. Compositions exhibit multiple functions that are useful for the treatment of pain and inflammation. Compositions of the invention inhibit the activity of COX-2 to a greater extent than COX-1. Compositions of the invention also inhibit the nuclear accumulation of NF-kappaB and thus inhibit the expression of a number of proinflammatory molecules including COX-2.

### TECHNICAL FIELD OF THE INVENTION

This invention relates generally to the field of using botanical extracts for ameliorating pain. More specifically, the invention provides compositions of botanical extracts and methods for their preparation and use in the treatment of pain. More particularly, the present invention relates to a method of decreasing or preventing pain associated with diseases, trauma or other conditions by administering botanical compositions of the present invention

### BACKGROUND OF THE INVENTION

Sensations that are unpleasant, intense, or distressing are described as painful. Pain is not homogeneous, however, and comprises three categories: physiological, inflammatory, and neuropathic pain. Multiple mechanisms contribute, each of which is subject to or an expression of neural plasticity—the capacity of neurons to change their function, chemical profile, or structure.

Over one-third of the world's population suffers from persistent or recurrent pain, costing the American public alone approximately $100 billion each year in health care, compensation, and litigation (Loeser, J. D., Butler, S. H., Chapman, C. R. & Turk, K. C., eds. (2001) Bonica's Management of Pain (Lippincott, Philadelphia)). Chronic pain is associated with conditions such as back injury, migraine headaches, arthritis, herpes zoster, diabetic neuropathy, temporomandibular joint syndrome, and cancer. Many of the currently available pain therapies are either inadequate or cause uncomfortable to deleterious side effects. Chronic pain results not just from the physical insult but also from a combination of physical, emotional, psychological, and social abnormalities. Because many pains persist after an insult is healed, the ongoing pain rather than the injury underlies the patient's disability. Untreated pain may become self-perpetuating because pain has immunosuppressive effects that leave patients susceptible to subsequent diseases.

Traumatic or nociceptive pain resulting from injury, surgery, inflammation, and including pain associated with diseases such as cancer, AIDS, arthritis, and herpes differs from neuropathic pain associated with diabetic neuropathy in that an external stimulus causes a normal sensory response to an insult or illness in the case of traumatic pain, whereas neuropathic pain results from injury to a portion of the nervous system and is typically not responsive to narcotic analgesics. Neuropathic pain often involves neural hypersensitivity and can persist without any overt external stimulus. (Goodman & Gilman's “The Pharmacologic Basis of Therapeutics”, 1996, p. 529, McGraw-Hill).

Major advances have occurred at levels spanning from molecular studies that have identified transduction proteins in nociceptors to cortical imaging studies which reveal how pain is experienced on a cognitive level (Woolf, C. J. & Salter, M. W. (2000) Science 288: 1765-1768). Two key lines of discovery have been (i) molecular/cellular transduction mechanisms and (ii) neuronal plasticity. (see Stucky C L, Gold M S, Zhang X. Mechanisms of pain. Proc Natl Acad Sci USA. 2001 Oct. 9; 98(21):11845-11846. Epub 2001 Sep. 18).

Physiological pain starts in the peripheral terminals of nociceptors with the activation of nociceptive transducer receptor/ion channel complexes, which generate depolarizing currents in response to noxious stimuli. Molecular genetic studies conducted in the past few years have identified specific molecules that are involved in the processes of pain transduction. Proteins called vanilloid receptors, e.g., VR1 and VRL1, allow detection of noxious heat (Caterina, M. J., Schumacher, M. A., Tominaga, M., Rosen, T. A., Levine, J. D. & Julius, D. (1997) Nature (London) 389: 816-824; Caterina, M. J., Rosen, T. A., Tominaga, M., Brake, A. J. & Julius, D. (1999) Nature (London) 398: 441-446). Without the VR1 receptor, one does not effectively detect noxious heat, particularly in the setting of inflammation (Caterina, M. J., Leffler, A., Malmberg, A. B., Martin, W. J., Trafton, J., Petersen-Zeitz, K. R., Koltzenburg, M., Basbaum, A. I. & Julius, D. (2000) Science 288: 306-313; Davis, J. B., Gray, J., Gunthorpe, M. J., Hatcher, J. P. Davey, P. T., Overend, P., Harries, M. H., Latcham, J., Clapham, C., Atkinson, K. , et al. (2000) Nature (London) 405: 183-187). The VR1 protein is a heat transducer which converts thermal energy into an electrical signal (action potentials) that is sent to the central nervous system, enabling detection of a stimulus as painfully hot. Recently, pain researchers have identified a number of transducer proteins that respond to extrinsic or intrinsic irritant chemical stimuli (VR1, DRASIC, P2X3) and are selectively expressed in sensory neurons molecules, which will clearly be key targets in developing pioneering pain therapies (McCleskey, E. W. & Gold, M. S. (1999) Annu. Rev. Physiol. 61: 835-856). There is a body of evidence relating activity at Group I mGluRs (mGluR1 and mGluR5) (M. E. Fundytus, CNS Drugs 15:29-58 (2001)) to pain processing.

Plasticity is a term used to refer to changes that occur in the established nervous system. Changes in neuronal structure; connections between neurons; and alterations in the quantity and properties of neurotransmitters, receptors, and ion channels can ultimately result in, increased functional activity of neurons in the pain pathway. Conversely, plasticity can decrease the body's own pain inhibitory systems, resulting ultimately in increased pain. Injury, inflammation, and disease can all cause neuronal plasticity and increased pain by means of increased excitatory or decreased inhibitory mechanisms. Plasticity can result in short-term changes that last minutes to hours, or long-term changes which may be permanent.

Nociceptors are a subpopulation of primary sensory neurons that are activated by “noxious” stimuli, i.e., stimuli that can produce tissue damage. Compelling evidence suggests that plasticity in nociceptors contributes substantially to the increased pain one feels in the presence of injury. Plasticity in nociceptors is critical for both the development and maintenance of plasticity in the central nervous system (Woolf, C. J. & Salter, M. W. (2000) Science 288: 1765-1768). That many receptors and ion channels recently identified are found specifically in nociceptors makes these proteins very good targets for eliminating pain without inducing side effects. Finally, the accessibility of the peripheral nervous system makes nociceptors a logical target for the development of novel therapeutic interventions.

Inflammation, inducible nitric oxide synthase (iNOS) activity and/or cytokine production has been implicated in a variety of diseases and conditions, including pain (Moore et al., “L-NG-nitro arginine methyl ester exhibits antinociceptive activity in the mouse,” Brit. J. Pharmacol., 102:198-202, 1991; Meller et al., “Production of endogenous nitric oxide and activation of soluble guanylate cyclase are required for N-methyl-D-aspartate-produced facilitation of the nociceptive tail-flick reflex,” Eur. J. Pharmacol., 214:93-96, 1992.; Lee et al., “Nitric oxide mediates Fos expression in the spinal cord induced by mechanical noxious stimulation,” NeuroReport, 3:841-844, 1992) and migraine (Olesen et al., “Nitric oxide is a key molecule in migraine and other vascular headaches,” Trends Pharmacol Sci., 15:149-153, 1994).

Nitric oxides (NOs) and prostaglandins (PGs) are well known proinflammatory mediators in the pathogenesis of inflammation. (Vane J. R., et al., Proc. Natl. Acad. Sci. U.S.A., 91, 2046-2050 (1994)). NO is synthesized by the three isoforms of nitric oxide synthase (NOS); neuronal NOS (nNOS), endothelial NOS (eNOS), and inducible NOS (iNOS). Although nNOS and eNOS are constitutively expressed, iNOS is expressed in response to interferon-gamma, lipopolysccharide (LPS), and a variety of proinflammatory cytokines. (Yun H. Y., Dawson T. T., Crit. Rev. Neurobiol., 10, 291-316 (1996)) A number of studies have shown that the chronic phase of inflammation in particular, is correlated with an increase in iNOS activity. (Miller M. J., Grisham M. B., Mediators Inflamm., 4, 387-396 (1995)).

Cyclooxygenase (COX) is involved in the inflammatory process and catalyzes the rate-limiting step in the synthesis of prostaglandins from arachidonic acid. COX exists in two isoforms; COX-1 and COX-2. (Funk C. D., et al., FASEB J., 5, 2304-2312 (1991)). COX-1 is expressed constitutively in most tissues and appears to be responsible for maintaining normal physiological functions whereas COX-2 is detected in only certain types of tissues and is induced transiently and up-regulated by various pro-inflammatory agents, including lipopolysaccharide, cytokines, and growth factors. (Hinz B., Brune K., J. Pharmacol. Exp. Ther., 300, 367-375 (2002).)

Endotoxin (bacterial lipopolysaccharide, LPS) is a major inflammatory molecule that triggers the production of proinflammatory cytokines such as TNF-alpha in various cell types. TNF-alpha plays a key role in the induction and perpetuation of inflammation in autoimmune reactions by activating T cells and macrophages, and by up-regulating other proinflammatory cytokines and endothelial adhesion molecules. (Beutler B., Cerami A., Ann. Rev. Immunol., 7, 625-655 (1989)).

TNF-alpha and LPS are known to activate transcription factors such as nuclear factor-kappa B (NF-κB). NF-κB is a member of the rel family of transcription factors and plays a key role in the regulation of inflammatory response, apoptosis and tumorigenesis. NFκB is activated by a wide variety of different stimuli such as pro inflammatory cytokines, oxidant free radicals, inhaled particles, ultraviolet radiation and bacterial or viral products. NF-κB is associated with the expression of pro-inflammatory genes during the onset of inflammation and with the expression of anti-inflammatory genes during the resolution of inflammation. Inhibition of NF-κB at the onset of inflammation results in decreased inflammatory response. (Lawrence et al Nature Medicine 7:1291 (2001), Transcription factors belonging to the NF-κB family regulate a range of genes that mediate inflammation and cell survival. (Farrow B., Evers B. M., Surg. Oncol., 10, 153-169 (2002)).

NF-κB exists in most cells as homodimeric or heterodimeric complexes containing p50 and p65 subunits, and remains inactive in the cytoplasm in association with the NF-kB inhibitory protein IκB. (Barnes P J, Karin M. Nuclear factor kappa B. A pivotal transcription factor for chronic inflammatory diseases. New Engl J Med 1997; 336: 1066-1071). Signaling cascades initiate phosphorylation and subsequent degradation of IkB protein. Upon stimulation, several types of kinases belonging to the mitogen-activated protein kinase (MAPK) family cause the phosphorylation and degradation of its Inhibitory kappa B Protein (IκB). The NFκB protein is freed from the inhibitor and translocates to the nucleus where it binds to its specific DNA motifs and initiates transcription of genes. The NF-κB increases the expression of genes encoding pro-inflammatory mediators, such as iNOS, COX-2, TNF-alpha, interleukin (IL)-6 and -8, and others. (see Imbert V., et al., Cell, 86, 787-798 (1996))

The therapeutic objective of most pain therapy is to alleviate the symptoms of pain regardless of cause. Nociceptive pain has been traditionally managed by administering non-opioid analgesics, such as acetylsalicylic acid, choline magnesium trisalicylate, acetaminophen, ibuprofen, fenoprofen, diflusinal, and naproxen; or opioid analgesics, including morphine, hydromorphone, methadone, levorphanol, fentanyl, oxycodone, oxymorphone, and nonsteroidal anti-inflammatory drugs (NSAIDS) such as aspirin, ibuprofen and cyclooxygenase inhibitors. In addition to the above-listed treatments, neuropathic pain, which can be difficult to treat, has also been treated with anti-epileptics (e.g. gabapentin, carbamazepine, valproic acid, topiramate, phenytoin), NMDA antagonists (e.g. ketamine, dextromethorphan), topical lidocaine (for post-herpetic neuralgia), and tricyclic antidepressants (e.g. fluoxetine, sertraline and amitriptyline). Current pain-control therapies also include the use of ion channel blockers such as lidocaine and novocaine.

In studies investigating the balance between the pro-oxidative and antioxidative defense system after repeated painful stimulation in rats and the efficacy of the administration of different antioxidants (vitamins C, E, A, and selenium), analgesics (acetylsalicylic acid, morphine), and their combinations were found to normalize both the oxidative stress and functional indicators of pain. Administration of antioxidants in pain treatment may be employed to decrease the doses of analgesics and to prevent the negative impact of reactive oxygen species on nociception. (Rokyta R, Holecek V, Pekarkova I, Krejcova J, Racek J, Trefil L, Yamamotova A. Free radicals after painful stimulation are influenced by antioxidants and analgesics, Neuroendocrinol Lett. 2003 October; 24(5):304-309.)

These therapies all have limitations. Opioids can cause tolerance, dependence, constipation, respiratory depression and sedation. NSAIDS have gastrointestinal side effects, can increase bleeding time, and are not effective in the treatment of severe pain. In the case of non-selective sodium channel blockers, central nervous system (CNS) side effects, cardiovascular side effects and corneal damage have been reported after use. Given the above limitations to currently known pain-control therapies, a need still exists for better pain-treatment methods.

### SUMMARY OF THE INVENTION

The present invention provides novel compositions, extracts and compounds comprising botanical extracts and their methods for manufacture and preparation. Use of such compounds in the prevention and reduction of pain are also provided as are methods for preparation and formulation of the compositions as well as methods for treatment using the compositions of this invention.

The compositions comprise therapeutically effective amounts of two or more of an extract of Ganoderma lucidum, an extract of Salvia miltiorrhiza and an extract of Scutellaria barbata; and optionally a therapeutically effective amount of an extract of Hippophae rhamnoides. Whereas there are reports of health benefiting effects of these individual botanicals, the synergistic effects of their use in combination therapy, as disclosed in this invention is novel.

The present invention relates to a method for reducing pain in a mammal in need of such treatment comprising administering a therapeutically effective amount of a compositions in combination with a pharmaceutically acceptable carrier.

The compositions of the present invention can be used alone to treat pain. The compositions of the present invention can also be used in conjunction with other therapeutic agents or adjunctive therapies commonly used to treat pain, thus enhancing the therapeutically desired effect of pain reduction

The compositions of the present invention comprise natural compounds that exhibit one or more properties of reducing inflammation, anti-oxidant activity, reducing nociceptive pain including tissue injury-induced pain and inflammatory pain, reducing neuropathic pain caused by damage to the peripheral or central nervous system and maintained by aberrant somatosensory processing.

While some compounds of the present invention have been known to demonstrate health benefits when administered individually, the present invention relates to novel combinations of natural compounds that demonstrate the properties of the compositions when administered as specified combinations. In general, the specific compositions of the present invention exhibit synergistic enhancement of their efficacies when administered in combination.

The compositions of the present invention act through multiple mechanisms for their anti-inflammatory and anti-nociceptive effects. The compositions exhibit direct inhibition of the COX-2 enzyme associated with the inflammatory process. The compositions also inhibit NF-κB activity which is involved in the expression of genes encoding pro-inflammatory mediators, such as iNOS, COX-2, TNF-alpha, interleukin (IL)-6 and -8, and others. A further inhibitory effect of the compositions of the present invention on the phosphorylation and degradation of IκB in a concentration-dependent manner suggests a further mechanism for modulation of NF-κB activity. The compositions of the present invention can reduce the amount of COX-2 enzyme expressed in cells by inhibiting NF-κB activity while directly inhibiting activity of the COX-2 enzyme already present in the cells.

The present invention and other objects, features, and advantages of the present invention will become further apparent in the following Detailed Description of the Invention and the accompanying Figures and embodiments.

### BRIEF DESCRIPTION OF THE FIGURES

FIG. 1 shows an extraction platform for botanical extracts.

FIG. 2 shows combination index (CI) values for the inhibition of COX-2 enzyme activity by ethyl acetate (upper panel) and methylene chloride (lower panel) extracts of the individual botanicals Ganoderma lucidum (#9), Scutellaria barbata (#15), and Salvia miltiorrhiza (#14) and combinations thereof.

FIG. 3 shows combination index (CI) values for the inhibition of COX-1 and COX-2 enzyme activities by ethyl acetate extracts of the individual botanicals Ganoderma lucidum (#9), Scutellaria barbata (#15), and Salvia miltiorrhiza (#14) and combinations thereof.

FIG. 4 shows the ratio of the potencies of inhibition of COX-2 over inhibition of COX-1 by ethyl acetate extracts (#0401) of the individual botanicals Ganoderma lucidum (#9), Scutellaria barbata (#15), and Salvia miltiorrhiza (#14) and combinations thereof.

FIG. 5 shows the potencies for inhibition of COX-2 and COX-1 by ethyl acetate extracts (#0401) of the individual botanicals Ganoderma lucidum (#9), Scutellaria barbata (#15), and Salvia miltiorrhiza (#14) and combinations thereof.

FIG. 6A shows the effects of extracts of the individual botanicals Ganoderma lucidum (#9), Scutellaria barbata (#15), and Salvia miltiorrhiza (#14) and combinations thereof on inhibition of COX-2 and COX-1 activities in vitro. FIG. 6B shows the relative inhibitions of COX-2 and COX-1 activities in vitro by the compositions.

FIGS. 7A and 7B shows levels of the p50 subunit of NF-κB in nuclear extracts of human epithelial lung cells (A549) subjected to the presence of 1× and 3× IC₅₀ of a composition (OMN54) comprising extracts of Ganoderma lucidum, Scutellaria barbata, and Salvia miltiorrhiza for 2 and 6 hours. FIG. 7C shows the effect of treatment with a composition (OMN54) comprising extracts of Ganoderma lucidum, Scutellaria barbata, and Salvia miltiorrhiza on the levels of p50 subunit of NF-κB in nuclear extracts of human epithelial lung cells (A549).

FIG. 8 shows the effects of ethyl acetate extracts (#0401) of the individual botanicals Ganoderma lucidum (#9), Scutellaria barbata (#15), and Salvia miltiorrhiza (#14) and combinations thereof on the body weight of SCID mice.

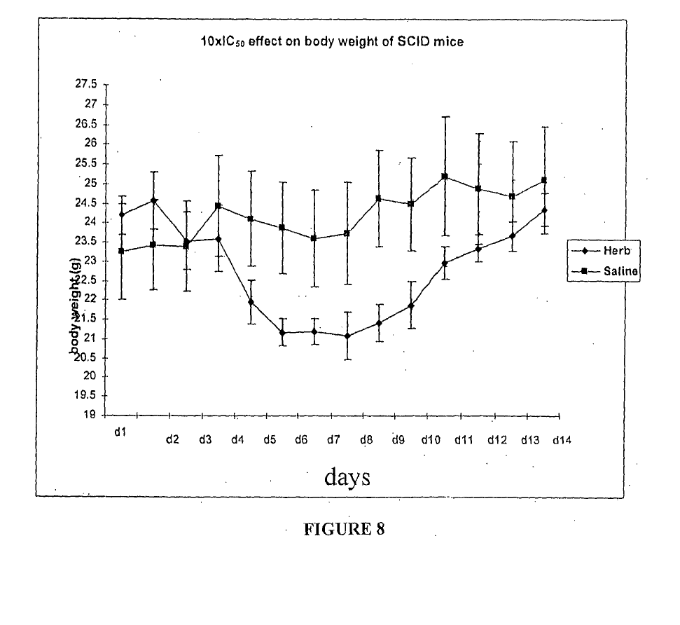

### DETAILED DESCRIPTION OF THE INVENTION

The present invention provides novel methods and compositions for use in treating pain in an individual. The present invention relates to a novel discovery that botanical extract-based compositions can effectively inhibit pain and be substantially nontoxic when administered to an individual. The composition comprises extracts of Ganoderma lucidum, Scutellaria barbata, Salvia miltiorrhiza, and optionally, Hippophae rhamnoides (sea buckthorn)

The term “pain” is used herein to represent all categories of physical pain. This includes traumatic pain resulting from injury, surgery or inflammation as well as pain associated with diseases such as cancer, AIDS, arthritis, and herpes. Pain can be associated with neuropathy such as diabetic neuropathy, causalgia, brachial plexus avulsion, occipital neuralgia, fibromyalgia, vulvodynia, prostadynia, pelvic pain, gout, and other forms of neuralgia, such as neuropathic and idiopathic pain syndromes. Specific organ- or site-localized pain, such as headache, ocular and corneal pain, bone pain, urogenital pain, heart pain, skin/burn pain, lung pain, visceral (kidney, gall bladder, etc.) pain, joint pain, dental pain and muscle pain are included in this invention. The general term “pain” also covers pain symptoms of varying severity, i.e. mild, moderate and severe pain, as well as those of acute and chronic pain.

As shown herein, pharmaceutical compositions containing compounds of the present invention have utility in the attenuation of pain signaling and therefore are useful for the treatment or prevention of pain. The method of the present invention using compositions of natural botanical extracts does not have many of the deficiencies and side effects of current commercial compounds and fulfills a need in treating pain by new modes or targets.

According to the present invention, the method of treating pain is in a subject in need of such treatment regardless of the cause or location of the bodily pain. The method according to a preferred embodiment of the present invention reduces pain with furanose-modified nucleoside polyphosphate derivatives and/or their dinucleotide analogs. The method comprises administering to a subject mammal, preferably a human, a pharmaceutical composition comprising an effective amount of the compositions of the present invention. The methods of the present invention are useful in the treatment of pain comprising traumatic pain, neuropathic pain, organ or tissue pain, or pain associated with diseases. An effective amount of said compound is an amount that leads to a reduction of nociception and/or ameliorates the symptoms of pain.

The method of the present invention alleviates the symptoms of pain regardless of the cause of the pain. Pain treatable by the present method includes traumatic pain, neuropathic pain, organ and tissue pain, and pain associated with diseases. Traumatic pain includes pain resulting from injury, post-surgical pain and inflammatory pain. Neuropathic pain includes neuropathic and idiopathic pain syndromes, and pain associated with neuropathy such as diabetic neuropathy, causalgia, brachial plexus avulsion, occipital neuralgia, fibromyalgia, gout, and other forms of neuralgia. Organ or tissue pain includes headache, ocular pain, corneal pain, bone pain, heart pain, skin/burn pain, lung pain, visceral pain (kidney, gall bladder, etc.), joint pain, dental pain, muscle pain, pelvic pain, and urogenital pain (e.g. vulvodynia and prostadynia). Pain associated with diseases includes pain associated with cancer, AIDS, arthritis, herpes and migraine. The present invention reduces pain of varying severity, i.e. mild, moderate and severe pain in acute and/or chronic modes.

In one embodiment, this method comprises administering a therapeutically effective amount of the composition to an individual (a mammal; and in a preferred embodiment, a human) bearing a tumor. In another embodiment, the method comprises administering a prophylactically effective amount of the composition to an individual to prevent tumor development (e.g., in an individual who is at high risk for developing tumor; or in an individual who is in remission, but at risk for recurrence).

The term “plant” as used herein refers to seeds, leaves, stems, flowers, roots, berries, bark, or any other plant parts that are useful for the purposes described. For certain uses, it is preferred that the underground portion of the plant, such as the root and rhizoma, be utilized. The leaves, stems, seeds, flowers, berries, bark, or other plant parts, also have medicinal effects and can be used for preparing tea and other beverages, cream, and in food preparation.

“Synergism” may be measured by combination index (CI). The combination index method was described by Chou and Talalay. (Chou, T.-C. The median-effect principle and the combination index for quantitation of synergism and antagonism, p. 61-102. In T.-C. Chou and D. C. Rideout (ed.), Synergism and antagonism in chemotherapy. Academic Press, San Diego, Calif. (1991); Chou, T.-C., and P. Talalay. Quantitative analysis of dose-effect relationships: the combined effects of multiple drugs on enzyme inhibitors. Adv. Enzyme Regul. 22:27-55 (1984)). A CI value of 0.90 or less is considered synergistic, with values of 0.85 being moderately synergistic and values below 0.70 being significantly synergistic. CI values of 0.90 to 1.10 are considered to be nearly additive and higher values are antagonistic.

TABLE 1
Synergism/antagonism as a function of CI values

| CI Value | Interpretation |
|---|---|
| >10 | Very strong antagonism |
| 3.3-10 | Strong antagonism |
| 1.45-3.3 | Antagonism |
| 1.2-1.45 | Moderate antagonism |
| 1.1-1.2 | Slight antagonism |
| 0.9-1.1 | Additive |
| 0.85-0.9 | Slight synergism |
| 0.7-0.85 | Moderate synergism |
| 0.3-0.7 | Synergism |
| 0.1-0.3 | Strong synergism |
| <0.1 | Very strong synergism |

It is noted that determination of synergy may be affected by biological variability, dosage, experimental conditions (temperature, pH, oxygen tension, etc.), treatment schedule and combination ratio. Synergism is measured as combination index (CI) values where values of 0.7 or less is considered to be significant levels of synergism.

### Botanicals

(i) Ganoderma lucidum (Reishi): Ganoderma lucidum was praised for its effect of increasing memory and preventing forgetfulness in old age reported in Shen Nong Ben Cao Jing vol. 1 as early as 456-536 AD. Research on mice using orally or topically administered Ganoderma lucidum suggests that Ganoderma lucidum has anti-inflammatory activity. Stavinoha, W., Satsangi, N., & Weintraub, S. (1995). Study of the anti-inflammatory efficacy of Ganoderma lucidum. In B.-K. Kim, & Y. S. Kim (Eds.), Recent Advances in Ganoderma lucidum research (pp. 3-7). Seoul Korea: The Pharmaceutical Society of Korea.

Applications of Ganoderma for (1) chemoprophylaxis of cancer in individuals at high risk for developing cancer (2) adjuvant use in the prevention of metastasis or recurrence of cancer (3) palliation of cancer related cachexia and pain and (4) adjunctive use with concurrent chemotherapy to reduce side-effects, maintain leukocyte counts and allow a more optimal dosing of chemo or radio therapeutics has been suggested. Chang, R (1994) Effective Dose of Ganoderma in Humans; Proceedings of Contributed Symposium 59A, B 5th International Mycological Congress, Vancouver: pp. 117-121. Since studies of human dosage were traditional and empiric a proper dose range of Ganoderma for therapy was calculated using this data and pharmacokinetic principals. The calculations suggested that a (1) Ganoderma dried fruit body dose of 0.5 to 1 g per day for health maintenance (2) 2 to 5 g per day if there is chronic fatigue, stress, auto immune, or other chronic health problems (3) 5 to 10 g per day for serious illness. Chang, R (1993) Limitations and Potential applications of Ganoderma and related fungal polyglycans in clinical ontology; First International Conference on Mushroom Biology and Mushroom products: 96.

(ii) Scutellaria barbata (Skullcap): Scutellaria barbata, a traditional Chinese medicine for liver, lung and rectal tumors, has been shown to inhibit mutagenesis, DNA binding and metabolism of aflatoxin B1 (AFB1) and cytochrome P450-linked aminopyrine N-demethylase. (Wong B. Y., et al. Eur J Cancer Prey 1993 July; 2(4):351-6; Wong B. Y., et al., Mutat Res. 1992 Jun. 1; 279(3):209-16). Scutellaria barbata is also capable of enhancing macrophage function in vitro and inhibiting tumor growth in vivo. (Wong B. Y., et al. Cancer Biother Radiopharm 1996 February; 11(1):51-6).

This botanical contains vitamins C and E as well as calcium, potassium, magnesium, iron, zinc scutellarin, volatile oil, tannin and bitter principles. The scutellarin acts on the central nervous system. Scutellarin, an active ingredient from Scutellaria barbata has been purified by liquid chromatography. (Wenzhu Zhang; Duolong Di; Bo Wen; Xia Liu; Shengxiang Jiang, Determination of Scutellarin in Scutellaria barbata Extract by Liquid Chromatography—Electrochemical Detection, Journal of Liquid Chromatography & Related Technologies 26 (13): 2133-2140 (2003).

(iii) Salvia miltiorrhiza (Dan Shen): There are over 900 species of salvia and many of them have histories of medicinal uses. Dan shen is used in traditional Chinese medicine to promote blood circulation and to remove blood stasis. Bensky D, Gamble A Chinese botanical Medicine Materia Medica 1987 Eastland Press: Seattle. 384. It increases the activity of superoxide dismutase (SOD) in platelets, thus providing protection against pulmonary embolism and inhibition of platelet aggregation. Wang X, et al. “Effect of danshen injection on pulmonary thromboembolism and platelet free radical levels in mice”. Zhongguo Zhong Yao Za Zhi 1996; 21:558-60. Salvia miltiorrhiza has been shown to lower cholesterol, reduce endothelial damage and to inhibit lipid peroxidation in hypercholesterolemic animals. This inhibition of oxidation of LDL may reduce atherosclerosis. Wu Y J, et al. “Increase of vitamin E content in LDL and reduction of atherosclerosis in cholesterol-fed rabbits by a water-soluble antioxidant-rich fraction of Salvia miltiorrhiza.” Arterioscler Thromb Vasc Biol 1998; 18:481-6. A Salvia miltiorrhiza constituent has been found to inhibit noradrenaline-induced contraction of the aortic strips through reduction in Ca²⁺ mobilization. This vasodilatory activity may explain the traditional use of Salvia miltiorrhiza in hypertension. Nagai M, et al. “Vasodilator effects of des (alpha-carboxy-3,4-dihydroxyphenethyl)lithospermic acid (8-epiblechnic acid), a derivative of lithospermic acids in salviae miltiorrhizae radix” Biol Pharm Bull 1996; 19:228-32. Salvia miltiorrhiza has been shown to have a markedly superior effect to nitroglycerin, with a more persistent action and better improvement of cardiac function. Bai Y R, Wang S Z. “Hemodynamic study on nitroglycerin compared with Salvia miltiorrhiza” Zhongguo Zhong Xi Yi Jie He Za Zhi 1994; 14:24-5, 4.

Salvia miltiorrhiza is also the top ingredient in Dan Shen Compound. Dan Shen Compound comprises four important botanicals for the improvement of peripheral circulation and general wellbeing. The actions of Crataegus laevigata are enhanced by the Chinese botanical Salvia miltiorrhiza (Dan Shen), the Indian botanical Coleus forskohlii and Valeriana officinalis. Chinese botanical medicine utilizes Salvia miltiorrhiza for women's irregularities, abdominal pain, insomnia, hives, hepatitis and mastitis.

(iv) Hippophae rhamnoides (sea buckthorn): Sea buckthorn seed oil contains a high content of the two essential fatty acids, linoleic acid and α-linolenic acid, which are precursors of other polyunsaturated fatty acids such as arachidonic and eicosapentanoic acids. The oil from the pulp/peel of sea buckthorn berries is rich in palmitoleic acid and oleic acid (Chen et al. “Chemical composition and characteristics of sea buckthorn fruit and its oil.” Chem. Ind. Forest Prod. (Chinese) 10 (3), 163-175). The increase in the level of a-linolenic acid in plasma lipids showed a clear improving effect on AD symptoms (Yang et al. J Nutr Biochem. 2000 Jun. 1; 11(6):338-340). These effects of α-linolenic acid may have been due to both changes in the eicosanoid composition and other mechanisms independent of eicosanoid synthesis (Kelley 1992, α-linolenic acid and immune response. Nutrition, 8 (3), 215-2).

Anti-oxidant and immunomodulatory properties of sea buckthorn (Hippophae rhamnoides) has been demonstrated using lymphocytes as a model system. (Geetha et al. J Ethnopharmacol 2002 March; 79(3):373-8). The antiulcerogenic effect of a hexane extract from Hippophae rhamnoides has also been demonstrated. (Suleyman H et al., Phytother Res 2001 November; 15(7):625-7). Radioprotection by a botanical preparation of Hippophae rhamnoides against whole body lethal irradiation in mice suggests free radical scavenging, acceleration of stem cell proliferation and immunostimulation properties. (Goel H C et al., Phytomedicine 2002 January; 9(1):15-25)

(v) Camellia sinensis (Green tea): Dried leaves from the Camellia sinensis plant is processed into three types of tea: oolong tea, black tea, and green tea. Green tea extract is a bioflavonoid-rich, potent extract which is used primarily for fighting free radicals. It has a high content of polyphenols, which are a Type of bioflavonoids. In making green tea, the tea leaves are stabilized by moist or dry heat which destroys the enzyme polyphenoloxidase and thus, prevents oxidation of polyphenols. These polyphenols are the main biologically active ingredients in green tea. In preferred embodiments, the green tea is Dragon Well tea or Lung Ching tea.

The polyphenols in green tea are catechins, with multiple linked ring-like structures. Polyphenols are a form of bioflavonoids with several phenol groups. They control both taste and biological action. Catechins, a chemical group of polyphenols possessing antioxidant properties (protecting cells from free radical-mediated damage), include epigallocatechin-3 gallate (EGCG), epigallocatechin, and epicatechin-3-gallate. Recently, ECGC has been shown to be an inhibitor of urokinase (Jankun et al., 1997, Nature 387:561), and quinol-oxidase; enzymes that may be crucial for growth of tumor cells. Epigallocatechin-3 gallate (EGCG) also protects against digestive and respiratory infections.

Ganoderma lucidum, Scutellaria barbata, Salvia miltiorrhiza, and Hippophae rhamnoides (sea buckthorn), and Camellia sinensis (green tea) have been used individually for health promoting and therapeutic purposes. Novel tumor inhibiting, immune boosting, inflammation reducing and anti-oxidative properties observed for compositions comprising extracts of Ganoderma lucidum, Scutellaria barbata, and Salvia miltiorrhiza and, optionally, Hippophae rhamnoides (sea buckthorn) and Camellia sinensis (green tea) and the synergistic effects demonstrated by novel combinations of two or more of these extracts used in the method according to the present invention are a likely result of combinations of one or more of saponins, flavonoids and polyphenols present in the extracts.

### Compositions

The compositions are standardized based on specific activities of defined properties which allows for very effective quality control based on standardized IC₅₀ based combinations. As discussed elsewhere in this application specific extraction procedures further facilitate the standardization of the compositions.

The compositions comprise botanical preparations extracted with hot water and organic solvents which allow convenient (e.g., oral) drug delivery.

The compositions of the present invention can be in any form which is effective, including, but not limited to dry powders, grounds, emulsions, extracts, and other conventional compositions. To extract or concentrate the effective ingredients of The compositions, typically the plant part is contacted with a suitable solvent, such as water, alcohol, methanol, or any other solvents, or mixed solvents. The choice of the solvent can be made routinely, e.g., based on the properties of the active ingredient that is to be extracted or concentrated by the solvent. Preferred active ingredients of The compositions crenulata include, but are not limited to, salidroside, tyrosol, β-sitosterol, gallic acid, pyrogallol, crenulatin, rhodionin, and/or rhodiosin. These ingredients can be extracted in the same step, e.g., using an alcoholic solvent, or they may be extracted individually, each time using a solvent which is especially effective for extracting the particular target ingredient from the plant. In certain embodiments, extraction can be performed by the following process: Milling the selected part, preferably root, to powder. The powder can be soaked in a desired solvent for an amount of time effective to extract the active agents from the compositions. The solution can be filtered and concentrated to produce a paste that contains a high concentration of the constituents extracted by the solvent. In some cases, the paste can be dried to produce a powder extract of The compositions crenulata. The content of active ingredient in the extract can be measured using HPLC, UV and other spectrometry methods.

The compositions of the present invention can be administered in any form by any effective route, including, e.g., oral, parenteral, enteral, intraperitoneal, topical, transdermal (e.g., using any standard patch), ophthalmic, nasally, local, non-oral, such as aerosol, inhalation, subcutaneous, intramuscular, buccal, sublingual, rectal, vaginal, intra-arterial, and intrathecal, etc. It can be administered alone, or in combination with any ingredient(s), active or inactive, including in a medicinal form, or as a food or beverage additive.

In preferred embodiments of the invention, the compositions are administered orally in any suitable form, including, e.g., whole plants, powdered or pulverized plant materials, extracts, pills, capsules, granules, tablets, lozenges, aqueous or oily suspensions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery. profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In yet another embodiment, the compositions can be delivered in a controlled-release system or sustained-release system (see, e.g., Langer, Science 249:1527-1533 (1990)). In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)).

The present compositions can optionally comprise a suitable amount of a pharmaceutically acceptable excipient so as to provide the form for proper administration to the mammal. The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule (see e.g., U.S. Pat. No. 5,698,155). Other examples of suitable pharmaceutical excipients are described in Remington's Pharmaceutical Sciences 1447-1676 (Alfonso R. Gennaro ed., 19th ed. 1995), incorporated herein by reference. Examples of suitable carriers are well known in the art and can include, but are not limited to, any of the standard pharmaceutical carriers such as a phosphate buffered saline solutions, phosphate buffered saline containing Polysorb 80, water, emulsions such as oil/water emulsion and various type of wetting agents. Other carriers may also include sterile solutions, tablets, coated tablets pharmaceutical and capsules. Typically such carriers contain excipients such as such as starch, milk, sugar, certain types of clay, gelatin, stearic acid or salts thereof, magnesium or calcium stearate, talc, vegetable fats or oils, gums, glycols. Such carriers can also include flavor and color additives or other ingredients. Compositions comprising such carriers are formulated by well known conventional methods. Generally excipients formulated with the compositions are suitable for oral administration and do not deleteriously react with it, or other active components.

Suitable pharmaceutically acceptable carriers include but are not limited to water, salt solutions, alcohols, gum arabic, vegetable oils, benzyl alcohols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as microcrystalline cellulose, citric acid, dextrin, dextrose, liquid glucose, lactic acid, lactose, magnesium chloride, potassium metaphosphate, starch, and the like.

The compositions can also be formulated with other active ingredients, such as anti-oxidants, vitamins (A, C, ascorbic acid, B's, such as B1, thiamine, B6, pyridoxine, B complex, biotin, choline, nicotinic acid, pantothenic acid, B12, cyanocobalamin, and/or B2, D, D2, D3, calciferol, E, such as tocopherol, riboflavin, K, K1, K2). Preferred compounds, include, e.g. creatine monohydrate, pyruvate, L-Carnitine, α-lipoic acid, Phytin or Phytic acid, Coenzyme Q10, NADH, NAD, D-ribose, amino acids such as L-glutamine, Lysine, chrysin; pre-hormones such as 4-androstenedione, 5-androstenedione, 4(or 5-)androstenediol, 19-nor-4 (or 5-)-androstenedione, 19-nor-4 (or 5-)-androstenediol, Beta-ecdysterone, and 5-Methyl-7-Methoxy Isoflavone. Preferred active ingredients include, e.g., pine pollen, fructus lycii, Hippophae rhamnoides, Ligusticum, Acanthopanax, Astragalus, Ephedra, codonopsis, polygola tenuifolia Willd, Lilium, Sparganium, ginseng, panax notoginseng, Garcinia, Guggle, Grape Seed Extract or powder, and/or Ginkgo Biloba.

Other plants and botanicals which can be formulated with the compositions of the present invention includes those mentioned in various text and publications, e.g., ES Ayensu, Medicinal Plants of West Africa, Reference Publications, Algonac, Mich. (1978); L. Boulos, Medicinal Plants of North Africa, Reference Publications Inc., Algonac, Mich. (1983); and N. C. Shah, Botanical Folk Medicines in Northern India, J. Ethnopharm, 6:294-295 (1982).

Other active agents include, e.g., antioxidants, anti-carcinogens, anti-inflammatory agents, hormones and hormone antagonists, antibiotics (e.g., amoxicillin) and other bacterial agents, and other medically useful drugs such as those identified in, e.g., Remington's Pharmaceutical Sciences (Mack Publishing Company, Alfonso R. Gennaro ed., 19th ed. 1995). A preferred composition of the present invention comprises, about 1%-100%, preferably about 20-70% of the botanical extract; and, optionally, a pharmaceutically-acceptable excipient.

The present invention relates to methods of administering the compositions, e.g., to provide antioxidant effects, to protect against oxidation, to provide anti-inflammatory effects, to prevent pain, to reduce pain, to reduce inflammation, and other conditions and diseases as mentioned herein.

By the term “administering,” it is meant that the compositions are delivered to the host in such a manner that it can achieve the desired purpose. As mentioned The compositions can be administered by an effective route, such as orally, topically, rectally, etc. The compositions can be administered to any host in need of treatment, e.g., vertebrates, such as mammals, including humans, male humans, female humans, primates, pets, such as cats and dogs, livestock, such as cows, horses, birds, chickens, etc.

An effective amount of the compositions are administered to such a host. Effective amounts are such amounts which are useful to achieve the desired effect, preferably a beneficial or therapeutic effect as described above. Such amount can be determined routinely, e.g., by performing a dose-response experiment in which varying doses are administered to cells, tissues, animal models (such as rats or mice in maze-testing, swimming tests, toxicity tests, memory tests as performed by standard psychological testing, etc.) to determine an effective amount in achieving an effect. Amounts are selected based on various factors, including the milieu to which the composition is administered (e.g., a patient with pain, animal model, tissue culture cells, etc.), the site of the cells to be treated, the age, health, gender, and weight of a patient or animal to be treated, etc. Useful amounts include, 10 milligrams-100 grams, preferably, e.g., 100 milligrams-10 grams, 250 milligrams-2.5 grams, 1 gm, 2 gm, 3 gm, 500 milligrams-1.25 grams. etc., per dosage of different forms of the compositions such as the botanical powder, botanical extract paste or powder, tea and beverages prepared to contain the effective ingredients of the compositions, and injections, depending upon the need of the recipients and the method of preparation.

### Compositions for Treatment of Pain

Compositions of the present invention comprise effective amounts of extracts of Ganoderma lucidum, Scutellaria barbata, Salvia miltiorrhiza, and optionally, Hippophae rhamnoides (sea buckthorn) that exhibit effects of inhibiting pain.

In one aspect of the invention, the composition comprises equal amounts of extracts of Ganoderma lucidum, Scutellaria barbata and Salvia miltiorrhiza. The dosage of the composition can be readily determined by one of skill in the art based on the effective concentrations of compositions shown to display the various properties described in this application. Compositions comprising different ratios of the individual extracts can similarly be determined.

The compositions are selected from combinations of extracts comprising two or more of Ganoderma lucidum, Scutellaria barbata, Salvia miltiorrhiza. Combinations of these compounds are shown to synergistically inhibit pain, reduce oxidation, and reduce inflammation.

In one aspect of the invention, the composition comprises equal amounts of extracts of Ganoderma lucidum, Scutellaria barbata and Salvia miltiorrhiza. The dosage of the composition can be readily determined by one of skill in the art based on the effective concentrations of compositions shown to display the various properties described in this application. Compositions comprising different ratios of the individual extracts can similarly be determined. Because pain inhibition can occur through a multitude of mechanisms, a composition may exhibit non-proportional degrees of pain inhibition at one concentration or ratios of combinations of extracts relative to other concentrations or ratios of combinations of extracts.

As known to those skilled in the art, the dosage may vary with the individual depending on the age, size, health, and metabolism of the individual, and related factors. The route of administration may be by any conventional route in which the composition can be safely and effectively delivered. A preferred route of administration is an oral route. The compositions are suited for convenient (oral) drug delivery. Botanicals are extracted with hot water and organic solvents (ethyl acetate ester, ethanol). The resulting composition may be administered in tablet/caplet/capsule form, or in a form in a pharmaceutically acceptable carrier (e.g., liquid, water, saline or other physiological solution, or gel).

Combinations of extracts comprising two or more of Ganoderma lucidum, Scutellaria barbata, Salvia miltiorrhiza are selected for the abilities to inhibit pain, reduce oxidation, and reduce inflammation. For quality control purposes IC₅₀ based compositions can be standardized based on specific activities of defined properties.

### Mechanisms of Pain Inhibition by Compositions of the Invention

Without being bound by theory, compositions of the present invention reduce pain by their effectiveness via one or more mechanisms. Compositions of the invention act though one or more of the following mechanisms: antioxidation, reducing nociceptive pain including tissue injury-induced pain and inflammatory pain, reducing neuropathic pain caused by damage to the peripheral or central nervous system and maintained by aberrant somatosensory processing,

Administration of antioxidants in pain treatment may be employed to decrease the doses of analgesics and to prevent the negative impact of reactive oxygen species on nociception. (Rokyta R, Holecek V, Pekarkova I, Krejcova J, Racek J, Trefil L, Yamamotova A. Free radicals after painful stimulation are influenced by antioxidants and analgesics, Neuroendocrinol Lett. 2003 October; 24(5):304-309.) The compositions show marked antioxidant activity.

Chronic pain can be classified as either nociceptive or neuropathic. Nociceptive pain includes tissue injury-induced pain and inflammatory pain such as that associated with arthritis. Neuropathic pain is caused by damage to the peripheral or central nervous system and is maintained by aberrant somatosensory processing. The compositions may inhibit the activity of both Group I mGluRs, mGluR1 and mGluR5, as a mechanism for pain inhibition. Inhibiting mGluR1 or mGluR5 reduces pain, as shown by in vivo treatment with antibodies selective for either mGluR1 or mGluR5, where neuropathic pain in rats was attenuated (M. E. Fundytus et al., NeuroReport 9:731-735 (1998)). It has also been shown that antisense oligonucleotide knockdown of mGluR1 alleviates both neuropathic and inflammatory pain (M. E. Fundytus et al., British Journal of Pharmacology 132:354-367 (2001); M. E. Fundytus et al., Pharmacology, Biochemistry & Behavior 73:401-410 (2002)). Alternately, the compositions may inhibit the vanilloid receptors such as VR1 to alter signals for pain processing.

The compositions of the present invention show marked anti-inflammation activity. The compositions are shown to reduce inflammation by inhibiting cyclooxygenases (COX-2) and reducing nuclear accumulation of the transcription factor NF-κB. The compositions show COX-2 inhibition (in preference over COX-1 by over 4×). This activity inhibits pain as COX-2 inhibitors are known as means for treating pain. Cyclooxygenase (COX) is a key enzyme in the biosynthesis of prostaglandin from arachidonic acid, and has two isotypes. COX-1 is responsible for producing the basal levels of prostaglandin needed for gastrointestinal tract homeostasis, whereas COX-2 is an inducible enzyme which is involved in inflammatory events. Well known nonsteroidal anti-inflammatory drugs (NSAIDs) such as aspirin, ibuprofen, and naproxen inhibit COX. Concentration-dependent inhibitory effects of the compositions of the present invention on LPS-induced PGE2 production, and COX-2 protein and mRNA expression indicate its effectiveness on reducing inflammation.

NF-κB is known to play a critical role in the regulation of genes involved in cell survival, and to coordinate the expressions of pro-inflammatory enzymes including iNOS, COX-2, and TNF-alpha. (Xie Q. W., et al., J. Biol. Chem., 269, 4705-4708 (1994); Chen F., et al., Biochem. Biophys. Res. Commun., 214, 839-846 (1995); Schmedtje J. F., Jr., et al, J. Biol. Chem., 272, 601-608 (1997); Roshak A. K., et al, J. Biol. Chem., 271, 31496-31501 (1996)) The reduction of nuclear accumulation of NF-κB by the compositions of the present invention suggests that the suppression of NF-κB could lead to the inhibition of iNOS and COX-2 proteins and iNOS, COX-2, and TNF-alpha mRNAs.

NF-κB is associated with an inhibitory subunit called IκB. NF-κB is present in the cytoplasm in an inactive form and is tightly controlled by IκB. However, when IκB is phosphorylated and is subsequently proteolysed, the translocation of NF-κB to the nucleus occurs, where it activates the transcriptions of NF-κB-responsible genes. (Henkel T., et al., Nature (London), 365, 182-185 (1993)). The inhibitory effect of the compositions of the present invention on the phosphorylation and degradation of IκB in a concentration-dependent manner suggests a further mechanism for the anti-inflammatory and anti-nociceptive effects of the composition. Thus the compositions of the present invention can reduce the amount of COX-2 enzyme produced in cells by inhibiting NF-κB activity and, also directly inhibit activity of the COX-2 enzyme already present.

### Uses of Compositions of the Invention for Pain Treatment or Prevention

In one embodiment, an effective amount of the compositions can be used to treat or prevent any condition treatable or preventable by inhibiting mGluR5. Examples of conditions that are treatable or preventable by inhibiting mGluR5 include, but are not limited to, pain, Parkinson's disease, parkinsonism, anxiety, a pruritic condition, and psychosis.

In another embodiment, an effective amount of the compositions can be used to treat or prevent any condition treatable or preventable by inhibiting mGluR1. Examples of conditions that are treatable or preventable, by inhibiting mGluR1 include, but are not limited to, pain, muscle spasm, migraine, vomiting, dyskinesia and depression.

The compositions can be used to treat or prevent acute or chronic pain. Examples of pain treatable or preventable using the compositions include, but are not limited to, cancer pain, central pain, labor pain, myocardial infarction pain, pancreatic pain, colic pain, post-operative pain, headache pain, muscle pain, pain associated with intensive care, arthritic pain, neuropathic pain, and pain associated with a periodontal disease, including gingivitis and periodontitis.

The compositions can also be used for inhibiting, preventing, or treating pain associated with inflammation or with an inflammatory disease in an animal. The pain to be inhibited, treated or prevented may be associated with inflammation associated with an inflammatory disease, which can arise where there is an inflammation of the body tissue, and which can be a local inflammatory response and/or a systemic inflammation. For example, the compositions can be used to inhibit, treat, or prevent pain associated with inflammatory diseases including, but not limited to: organ transplant rejection; reoxygenation injury resulting from organ transplantation (see Grupp et al., J. Mol. Cell Cardiol. 31:297-303 (1999)) including, but not limited to, transplantation of the heart, lung, liver, or kidney; chronic inflammatory diseases of the joints, including arthritis, rheumatoid arthritis, osteoarthritis and bone diseases associated with increased bone resorption; inflammatory lung diseases, such as asthma, adult respiratory distress syndrome, and chronic obstructive airway disease; inflammatory diseases of the eye, including corneal dystrophy, trachoma, onchocerciasis, uveitis, sympathetic ophthalmitis and endophthalmitis; chronic inflammatory diseases of the gum, including gingivitis and periodontitis; tuberculosis; leprosy; inflammatory diseases of the kidney, including uremic complications, glomerulonephritis and nephrosis; inflammatory diseases of the skin, including sclerodermatitis, psoriasis and eczema; inflammatory diseases of the central nervous system, including chronic demyelinating diseases of the nervous system, multiple sclerosis, AIDS-related neurodegeneration and Alzheimer s disease, infectious meningitis, encephalomyelitis, Parkinson's disease, Huntington's disease, amyotrophic lateral sclerosis and viral or autoimmune encephalitis; autoimmune diseases, including Type I and Type II diabetes mellitus; diabetic complications, including, but not limited to, diabetic cataract, glaucoma, retinopathy, nephropathy (such as microaluminuria and progressive diabetic nephropathy), polyneuropathy, mononeuropathies, autonomic neuropathy, gangrene of the feet, atherosclerotic coronary arterial disease, peripheral arterial disease, nonketotic hyperglycemic-hyperosmolar coma, foot ulcers, joint problems, and a skin or mucous membrane complication (such as an infection, a shin spot, a candidal infection or necrobiosis lipoidica diabeticorum); immune-complex vasculitis, and systemic lupus erythematosus (SLE); inflammatory diseases of the heart, such as cardiomyopathy, ischemic heart disease hypercholesterolemia, and atherosclerosis; as well as various other diseases that can have significant inflammatory components, including preeclampsia, chronic liver failure, brain and spinal cord trauma, and cancer.

The compositions can also be used for inhibiting, treating, or preventing pain associated with inflammatory disease that can, for example, be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

### EXAMPLES

Without further elaboration, it is believed that one skilled in the art can, using the preceding description, utilize the present invention to its fullest extent. The following examples are illustrative only, and not limiting of the remainder of the disclosure in any way whatsoever.

The following combinations of extracts were used throughout the examples: Ganoderma lucidum, Scutellaria barbata, and Salvia miltiorrhiza. In addition, the compositions of the invention may include, optionally, Panax Quinquefolium (Western ginseng), Camellia sinensis (green tea), and Hippophae rhamnoides (sea buckthorn).

### Example 1

### Methods for Preparation of Botanical Extracts

The compositions of the present invention may be administered as dried botanicals. Botanical preparations contain phytochemicals some of which are soluble in aqueous media while others are relatively more soluble in organic (alcohol, lipid) media. Different extraction methods were used and tested for the ability to extract effective ingredients from the botanicals. Extraction methods include: Hot Water extraction; Organic (lipid fraction) extraction; Organic (aqueous fraction) extraction; and Ethanol Extraction.

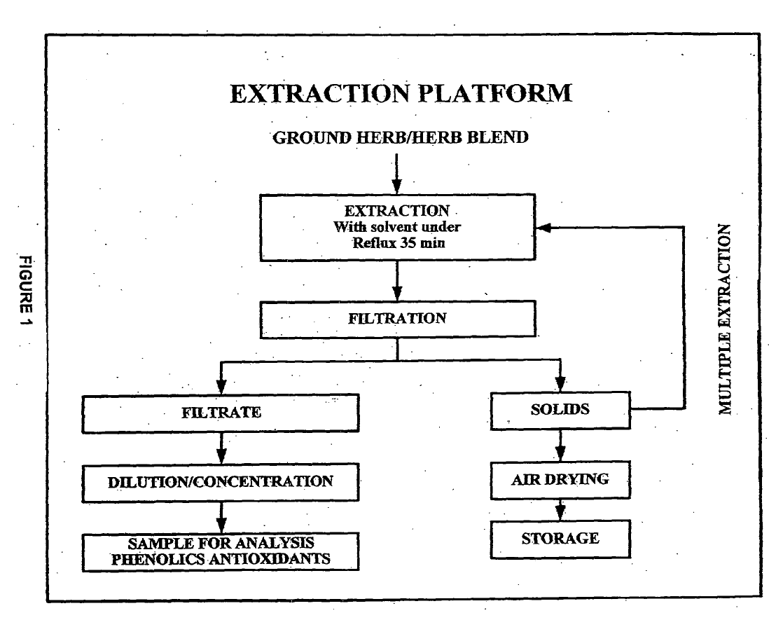

Products are prepared from botanicals using different solvents by the general extraction platform shown in FIG. 1A. In general, the botanicals are pre-screened for uniform size and quality by visual and other inspection means. The raw botanical material is extracted with the desired solvent. Preferably, the extraction process is carried out twice for each batch. The liquid extracts are evaporated to dryness. If needed, the solvent is removed and the dried extracts are blended as the final products. Optionally, the blends may be encapsulated for storage and delivery.

In the extraction schemes depicted in FIGS. 1B-1G, botanical or botanical blends were extracted with solvent (hot water, 80% ethanol, or ethyl acetate) under reflux for 30-60 minutes, separated by filtration to obtain a filtrate, and air dried for further analysis. The filtrates were combined, diluted or concentrated prior to determination of activities. Extraction procedures with hot water, 80% ethanol and chloroform/methanol are shown schematically in FIGS. 1B, 1C, and 1D respectively. Extraction procedures of botanical blends with hot water, 80% ethanol and hot water followed by 80% ethanol are illustrated in FIGS. 1E, 1F and 1G respectively. Extraction procedure of botanical blends with ethyl acetate is illustrated in FIG. 1H

### Example 2

### Cox-2 Inhibition by Extracts

Cyclooxygenase (Cox) is an enzyme naturally present in our body. Cox-2 is an enzyme that is necessary for inducing pain. Nonsteroidal anti-inflammatory drugs (NSAIDs) are widely used in treating pain and the signs and symptoms of arthritis because of their analgesic and anti-inflammatory activity. It is accepted that common NSAIDs work by blocking the activity of cyclooxygenase (COX), also known as prostaglandin GM synthase (PGHS), the enzyme that converts arachidonic acid into prostanoids. Recently, two forms of COX were identified, a constitutive isoform (COX-1) and an inducible isoform (COX-2) of which expression is up regulated at sites of inflammation (Vane, J. R.; Mitchell, J. A.; Appleton, I.; Tomlinson, A.; Bishop-Bailey, D.; Croxtoll, J.; Willoughby, D. A. Proc. Nat. Acad. Sci. USA, 1994, 91, 2046). COX-1 is thought to play a physiological role and to be responsible for gastrointestinal and renal protection. On the other hand, COX-2 appears to play a pathological role and to be the predominant isoform present in inflammation conditions. The COX-2 enzyme is specific for inflammation, and Cox2 inhibitors (such as Celebrex®, Vioxx®) have been approved by the FDA.

The inhibition of COX-2 is responsible for the anti-inflammatory effects of NSAIDS, while inhibition of COX-1 is responsible for the recognized toxicities of NSAIDs, including: a) peptic ulcers and the associated risks of bleeding, perforation and obstruction; b) prolonged bleeding time; and, c) renal insufficiency. Drugs that would selectively inhibit COX-2 are thus highly desirable since inflamed tissues could be targeted without disturbing the homeostatic functions of prostaglandins in noninflamed organs. Theoretically, then, selective COX-2 inhibition should preserve the anti-inflammatory efficacy without causing the associated toxicities of NSAIDs.

The anti-inflammatory assays for COX-2 inhibitory activity were conducted using prostaglandin endoperoxide H synthase-1 and -2 isozymes (PGHS-1, and -2) based on their ability to convert arachidonic acid to prostaglandins (PGs). The positive controls used in this experiment are aspirin, naproxen, and ibuprofen.

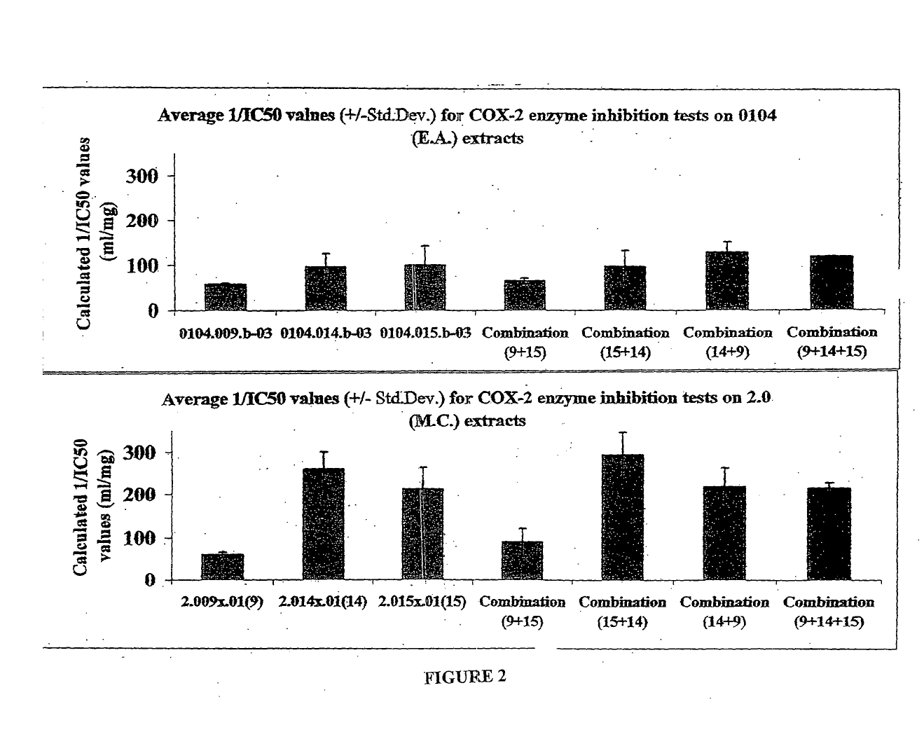

Combination index (CI) values for the inhibition of COX-2 enzyme activity by methylene chloride extracts of the individual botanicals Ganoderma lucidum (#9), Scutellaria barbata (#15), and Salvia miltiorrhiza (#14) and combinations thereof were measured. The inverse of the concentration of extract(s) that inhibited enzyme activity by 50% of maximum inhibition (as measured by heat inactivation) is shown in FIG. 2. The combination of Ganoderma lucidum (#9) and Salvia miltiorrhiza (#14) showed the most synergism as did the combination of all three botanicals.

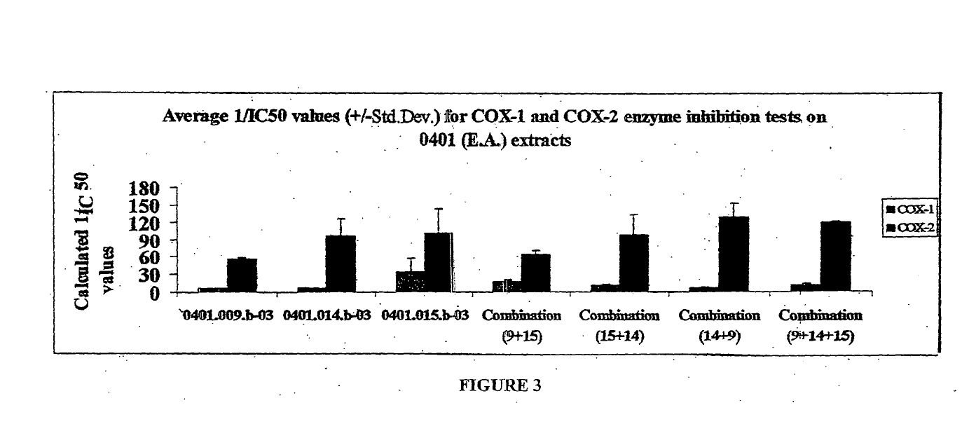

Combination index (CI) values for the inhibition of COX-2 enzyme activity by ethyl acetate extracts of the individual botanicals Ganoderma lucidum (#9), Scutellaria barbata (#15), and Salvia miltiorrhiza (#14) and combinations thereof were measured. The inverse of the concentration of extract(s) that inhibited enzyme activity by 50% of maximum inhibition (as measured by heat inactivation) is shown in FIG. 3. The combination of Ganoderma lucidum (#9) and Scutellaria barbata (#15) showed any significant synergism (CI˜0.6).

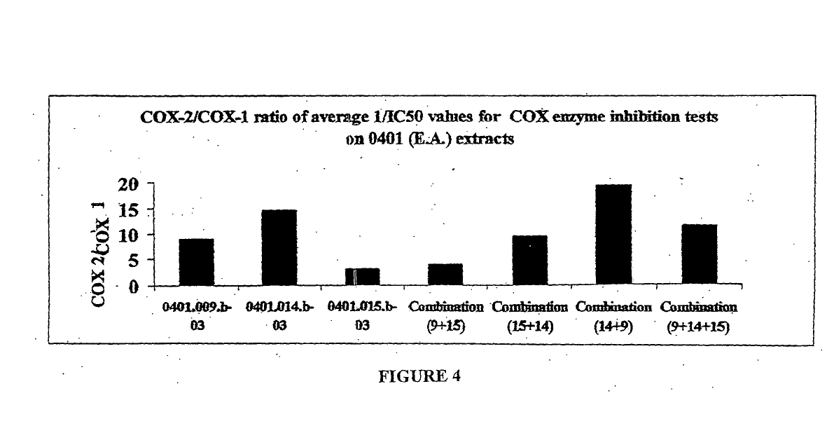

A preferred COX-2 inhibitor would exhibit greater inhibition of COX-2 over COX-1, which is responsible for gastrointestinal and renal protection. The ratio of the potencies of inhibition of COX-2 over inhibition of COX-1 by ethyl acetate extracts (#0401) of the individual botanicals Ganoderma lucidum (#9), Scutellaria barbata (#15), and Salvia miltiorrhiza (#14) and combinations thereof were measured and is shown in FIG. 4. The combinations shown were prepared by mixing two or more extracts in the ratios of their IC₅₀s for inhibiting either COX-1 or COX-2 activity. Thus different combination mixtures were used for COX-1 and COX-2 inhibition. The extract of Salvia miltiorrhiza (#14) was the most selective single agent and showed a 15-fold preference for COX-2 over COX-1. The combination of extracts of Ganoderma lucidum (#9) and Salvia miltiorrhiza (#14) was 19-fold more potent in inhibiting COX-2 over COX-1 a shown in FIG. 4.

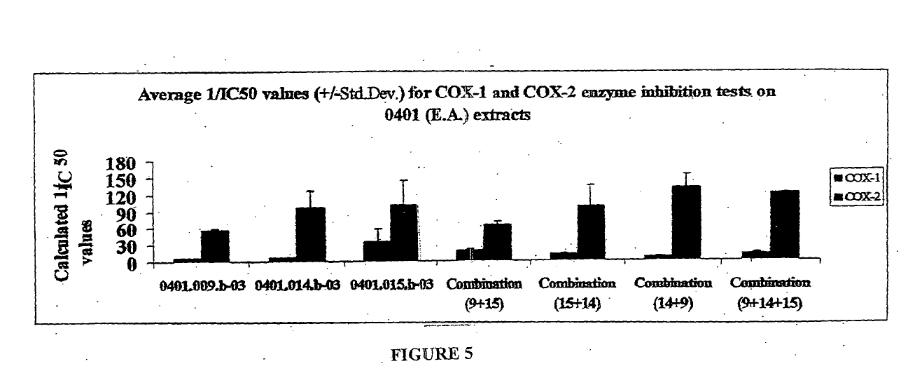

FIG. 5 shows the potencies for inhibition of COX-2 and COX-1 by ethyl acetate extracts (#0401) of the individual botanicals Ganoderma lucidum (#9), Scutellaria barbata (#15), and Salvia miltiorrhiza (#14) and combinations thereof Potency is represented as the inverse of the IC50 of each composition tested. Inhibition was measured by COX-1 and COX-2 ELISA assay kits (Cayman Chemical Co., Ann Arbor, Mich.). Salvia miltiorrhiza (#14) alone or in combination with Ganoderma lucidum (#9), or Ganoderma lucidum (#9) and Scutellaria barbata (#15) showed the most potency.

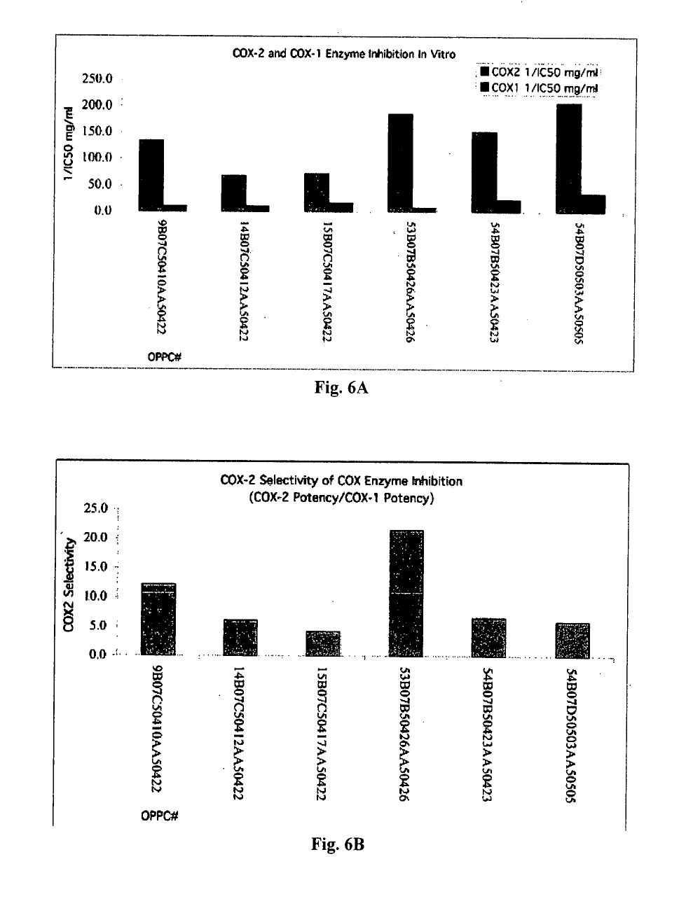

FIG. 6A shows the effects of extracts of the individual botanicals Ganoderma lucidum (#9), Scutellaria barbata (#15), and Salvia miltiorrhiza (#14) and combinations thereof on inhibition of COX-2 and COX-1 activities in vitro. FIG. 6B shows the relative inhibitions of COX-2 and COX-1 activities in vitro by the compositions. The compositions of the invention are able to directly inhibit COX activity with a 5-25 fold selectivity in inhibiting COX-2 over COX-1.

### Example 3

### Anti-Oxidant Activity of Extracts

Blends of botanical extracts comprising two or more of sea buckthorn berry, sea buckthorn leaf, Pq, Ganoderma lucidum, Salvia miltiorrhiza and Scutellaria barbata are tested for anti-oxidant property. Blend A comprised all 6 ingredients and Blends B-G specifically excluded one component at a time. Sea buckthorn leaf was found to be responsible for nearly 50% of the anti-oxidant activity of the entire blend.

Blends of hot water extracts comprising two or more of Ganoderma lucidum, Salvia miltiorrhiza and Scutellaria barbata are tested for anti-oxidant property expressed. The standard of comparison is Trolox (a water-soluble analog of vitamin E), and the relative anti-oxidant activity is defined as Trolox Equivalents (TE). The standard of comparison in is Quercetin (a flavonoid), and the relative anti-oxidant activity is defined as Quercetin Equivalents. Sea buckthorn leaf was found to be responsible for nearly 50% on the anti-oxidant activity of the entire blend under both systems of measurement.

### Example 4

### Measurement of Pain Reduction by Laser Algesimetry

Blends of botanical extracts comprising two or more of sea buckthorn berry, sea buckthorn leaf, Ganoderma lucidum, Salvia miltiorrhiza and Scutellaria barbata are tested for pain reduction property.

Objective and quantitative measurement of pain relief are performed by Laser-algesimetry. Laser algesimetry involves experimental induction of pain using a laser beam with constant power, short duration and individually adjusted intensity—just above individual pain threshold. Objective/quantitative measurement of inflicted pain is accomplished by analysis of the contingent event related Vertex-EEG changes (laser-induced somatosensory evoked potentials, LSEPs).

Analgesic properties of the compositions are demonstrated objectively and quantitatively by alterations of the SEP-parameters vs. placebo, primarily by reductions of amplitudes.

A CO₂-laser is used for stimulation. The major advantage versus other techniques used for sensory stimulation is, that thermo-nociceptors of A-delta (thinly myelinated) and C-fiber (nonmyelinated) type are selectively stimulated without any direct skin contact (high receptors specificity) by means of a CO₂-laser beam with a low depth of penetration due to its wave length being in the far infrared part of the spectrum (99% of the laser energy is absorbed in the skin layer, where the free nociceptor terminals are located and its heat-sensitive ionic channels are activated).

The two main EP-components are evaluated with regard to their complex peak-to-peak amplitude as well as with regard to the single N1-component, mainly reflecting “peripheral” effects and P2-component, mainly reflecting “central” effects in pain relief mechanisms. Analgesics of the peripheral type preferably depress the N1-amplitudes (Schaffler K, Wauschkuhn C H, Brunnauer H, Rehn D. Evaluation of the local anaesthetic activity of dimetindene maleate by means of laser algesimetry in healthy volunteers. Arzneimittelforschung. 1992 November; 42(11):1332-5), and to a lesser extent the P2-amplitudes. Analgesics of the central type depress preferentially the P2-amplitude (Schaffler 1991 Schaffler K, Wauschkuhn C H, Gierend M. Analgesic potency of a new anticonvulsant drug versus acetylsalicylic acid via laser somatosensory evoked potentials. Randomized placebo-controlled double-blind (5-way) crossover study. Arzneimittelforschung. 1991 April; 41(4):427-35.).

During stimulation, the subjects sit on a chair, with their arms resting on a table in front of them and their head fixed in an ophthalmologic forehead-chin rest for positioning and relaxation of neck muscles, in order to avoid myogenic artifacts. The laser shots are applied to the back. Since alterations in vigilance have an impact on EP amplitudes (Condes-Lara M, Calvo J M, Fernandez-Guardiola A. Habituation to bearable experimental pain elicited by tooth pulp electrical stimulation. Pain. 1981 October; 11(2):185-200.), there is a need for vigilance control during LSEP assessment. This is done by loading the subjects with a pursuit tracking task (PTT) which is performed for the entire period of recording LSEP. To avoid influences of external disturbing noise (distraction), the subjects wear earphones which produce “white noise” (sound pressure of 90 dBA).

Due to automatic artifact detection, evaluation of EEG data is optionally done on-line during registration and SEP-parameters are derived immediately after completion of data acquisition.

The LSEP-N1-/P2-peak-to-peak amplitudes are defined as the main target variables for the investigation of analgesic effects. Analgesic effects of active treatments with effective amounts of the compositions of this invention result in a reduction of the N1- and P2-amplitudes.

In addition to normal skin, laser stimulation of UV- and capsaicin-irritated skin is used as different models with inflammatory, neurogenic erythema for induction of pain and hyperalgesic states. The UV model is more related to the complete cyclo-oxygenase cascade (injuries and inflammation of acute and subchronic type), whereas capsaicin is more related to hyperalgesic (peripheral, central-spinal) neuropathic states.

### Example 5

### Reduction of NF-κB in Epithelial Lung Carcinoma A549 Cells by the Botanical Compositions

Proinflammatory cytokines such as IL1B and TNF-alpha play a major role in pain facilitation. These cytokines exert their actions through activation of NF-κB. Intrathecal administration of NF-κB inhibitors partially attenuated allodynia in several rat models and demonstrated that spinal cord NF-κB activation was involved, at least in part, in exaggerated pain states. (Ledeboer A et al., Eur. J. Neurosci. 2005 22:1977-1986). Spinal nerve injury was found to cause mechanical and thermal hyperalgesia in the hind paw in rat model and stimulated expression of NF-κB, TNF-alpha, IL-1β and 1L-10 in the brain. (Xie Wet al., Neurosci. Lett. 2006 393:255-259). Oxaprozin has strong analgesic qualities particularly useful in painful musculoskeletal conditions and exhibits inhibition of COX-1, COX-2, inhibition of NF-κB and metalloproteases. (Kean W F, Curr. Med. Res. Opin. 2004 20:1279-1290). IL-1β induced spinal COX-2 up-regulation and pain hypersensitivity following peripheral inflammation was mediated through the activation of the NF-κB-associated pathways. (Lee K M et al., Eur. J. Neurosci. 2004 19:3375-3381). Specific inhibition of IκB kinase reduces hyperalgesia in inflammatory and neuropathic pain models in rats. IκB kinase (IKK) inhibitors prevented the translocation of NF-κB in the spinal cord and up-regulation of NF-kappaB-responsive genes including COX-2, TNF-alpha and IL-1β. The inhibitors appeared to reverse thermal and mechanical hyperalgesia in a rat model. (Tegeder I et al., J. Neurosci. 2004 24:1637-1645). Endoneurial injection of a NF-κB decoy significantly alleviated thermal hyperalgesia and suppressed the expression of mRNA of the inflammatory cytokines, iNOS and adhesion molecules at the site of nerve injury suggesting that a perineural inflammatory cascade, that involved NF-κB, was involved in the pathogenesis of neuropathic pain. (Sakaue G et al., Neuroreport 2001 12:2079-2084).

The NFκB family consists of a number of structurally related members (p50, p52, RelA, RelB, RelC). These are dimeric (homo and hetero) complexes containing various combinations of these proteins. The members of the NFκB protein group, presently best characterized are the p50/p65 heterodimer and the p50/p50 homodimer complexes.

The botanical compositions comprising two or more of botanicals Ganoderma lucidum, Scutellaria barbata, and Salvia miltiorrhiza were assayed for the ability to reduce the constitutive concentration of p50 (one of the most abundant members of NF-κB family of nuclear transcription factors) in the nuclei of cells. Several methods are available for measuring NF-κB p50 levels in cells. Nuclear extracts can be prepared using the method described by Dignam et al. (Dignam, J. D., Lebovitz, R. M., and Roeder, R. G. (1983) Nucleic Acids Research 11:1475-1489). Alternatively, a commercially available kit can be used. (e.g., Panomics Nuclear Extraction Kit, Panomics Inc. Freemont, Calif.)

Electrophoretic mobility assay (EMSA): A nuclear extract of the relevant cell type can be used for blot or gel studies. One can show NF-κB p50 by Western blot analysis. The electrophoretic mobility assay (EMSA) measures the ability of active NF-κB to bind to specific DNA sequences. Changes in the mobility of DNA probes containing B sites can be assessed when incubated with nuclear extract. The nuclear extracts are mixed with double stranded ³²P-dATP oligonucleotide carrying the decameric NF-κB binding site. Electrophoresis through 5% polyacrylamide gel is carried out. When nuclei have been activated by a cytokine like IL-1, autoradiography shows that NF-κB p50 has shifted. Confirmation of the specificity is provided by incubation with specific antibody against Rel/NF-κB proteins that identify a super-shift. Control lanes are run in which (i) unlabelled probe serves as cold competitor and (ii) mutated oligonucleotides are used as specific DNA competitor (Hernandez-Presa M A, Gomez-Guerrero C, Egido J. In situ non-radioactive detection of nuclear factors in paraffin sections by Southwestern histochemistry. Kidney Int. 1999; 55: 209-214). EMSA is specific and reproducible but only semi-quantitative.

In one embodiment of the EMSA assay for NF-κB, nuclear extracts are prepared as described by Dignam et al. (Nucleic Acids Research 11:1475-1489 (1983)). ³²P-end-labeled double-stranded oligonucleotide probes used in this study comprised either wild type NF-κB oligonucleotide (sense: 5′-TGAGGGGACTTTCCCAGG-3′), or p50/p65 mutant oligonucleotide (sense: 5′-TGAGGCGACTTTCCCAGG-3′). The double-stranded NF-κB oligomers are used in nuclear protein-DNA binding reactions (20 μl volume) in which 1 μg poly dI:dC and 6 μg nuclear protein extract are incubated for 20 min at 4° C. prior to addition of 0.2 ng ³²P-labeled double-stranded oligonucleotide for 30 min at 4° C. The contents of each tube are electrophoresed on non-denaturing 4% polyacrylamide gels which are then dried and analyzed by autoradiography. Supershift assays are performed by incubating pre-assembled gel shift assay complexes containing 8 μg nuclear extract with either 2 μg rabbit normal IgG, 2 μg rabbit polyclonal anti-p65 NF-κB IgG or/and 2 μg rabbit polyclonal anti-p50 NF-κB IgG (Santa Cruz Biotechnology Inc, CA, USA) for 2 h at 4° C. before electrophoresis. The samples were then electrophoresed on 8% polyacrylamide gels (Pizzi M., et al. J Biol Chem 2002, 277(23):20717-20723).

Optical Biosensor assay for protein interactions: Alternatively, a BIACORE optical biosensor (Biacor International AB, Sweden) is used to determine activated NF-κB. Biotinylated NF-κB sense and antisense consensus sequences are hybridized and captured onto a streptavidin-coated sensor chip. Nuclear extract is passed over the captured sequence and, when activated NF-κB is present, a signal is generated.

ELISA: An oligonucleotide containing a NF-κB consensus binding site is immobilized on a 96-well plate. Activated NF-κB p50 from nuclear or whole-cell extracts will specifically bind to this oligonucleotide. The complex bound to the oligonucleotide is detected by antibody directed against the p50 subunit. An additional secondary HRP-conjugated antibody provides sensitive colorimetric readout easily quantified by spectrophotometry. An ELISA kit for NF-κB p50 assay is available from Panomics, Inc. (Freemont, Calif.). TransAM™ NF-κB kits (Active Motif Carlsbad, Calif.) are available with antibodies specific for the activated form of p50 and p65 subunit of the NFkB. Both are available in colorimetric and chemiluminescent versions.

SDS-PAGE and immunoblotting: 25 μg nuclear extracts prepared as described by Dignam et al. (Nucleic Acids Research 11:1475-1489 (1983)) are subjected to SDS-PAGE in 5-15% gradient gels at 120 V for 1.5 h. Proteins are transferred to nitrocellulose membranes which are individually incubated with 1:500 dilutions of rabbit anti-IκBα, -phospho-IκBα, -NF-κB p65, or -NF-κB p50 polyclonal IgG (Santa Cruz Biotechnology Inc, CA, USA) in 5% nonfat milk TBST for 24 h at 4° C. The filters are then incubated with 1:1000 dilutions of HRP-conjugated goat anti-rabbit IgG for 1 h at room temperature. The membrane is washed extensively before detection using chemiluminescence.

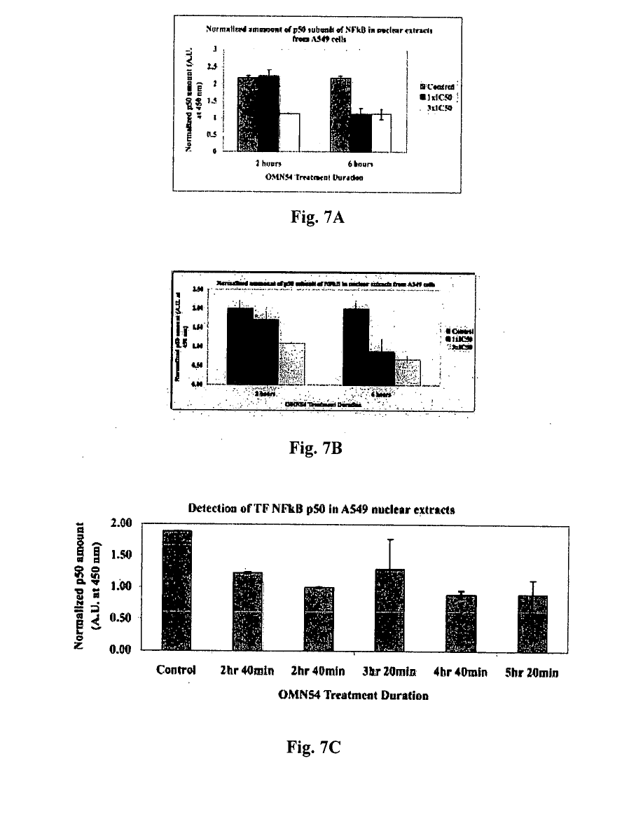

FIGS. 7A and 7B shows levels of the p50 subunit of NF-κB in nuclear extracts of human epithelial lung cells (A549) subjected to the presence of 1× and 3× IC₅₀ of a composition (OMN54) comprising extracts of Ganoderma lucidum, Scutellaria barbata, and Salvia miltiorrhiza for 2 and 6 hours. FIG. 7C shows the effect of treatment with a composition (OMN54) comprising extracts of Ganoderma lucidum, Scutellaria barbata, and Salvia miltiorrhiza on the levels of p50 subunit of NF-κB in nuclear extracts of human epithelial lung cells (A549). Significant reduction (˜2 fold) in the nuclear content of p50 subunit of NF-κB was observed following two to three hours of treatment of A549 cells with 1× IC₅₀ and 3× IC₅₀ of OMN54.

### Example 6

### Modulation of Degradation of IκB by the Botanical Compositions

Important modulators of NF-κB activation are the inhibitor proteins IκBα and IκBβ, which associate with (and thereby inactivate) NF-κB in vivo. Activation and nuclear translocation of NF-κB occurs following signal-induced phosphorylation of IκB, which leads to proteolysis via the ubiquitin pathway. For IκBα, the stimulus-induced phosphorylation at serines 32 and 36 renders the inhibitor a target for ubiquitination at lysines 21 and 22, resulting in degradation. Similarly, phosphorylation of IκBβ at serines 19 and 23 renders the inhibitor a target for ubiquitination at lysine 9. However, neither the site at which IκBs are recognized by the ubiquitin system, nor the component(s) of the ubiquitin system mediating IκB recognition have been identified. Degradation of a protein via the ubiquitin pathway proceeds by covalent attachment of multiple ubiquitin molecules to the protein substrate, followed by degradation of the targeted protein by the 26S proteasome complex. The ubiquitin pathway consists of several components that act in concert and in a hierarchical manner (for reviews, see Ciechanover, Cell 79:13, 1994; Hochstrasser, Curr. Op. Cell. Biol. 7:215, 1995; Jentsch and Schlenker, Cell 82:881, 1995; Deshaies, Trends Cell Biol. 5:428, 1995).

The present invention provides compositions and methods for modulating the activation of nuclear factor κB (NF-κB) by modulating ubiquitination of phosphorylated IκBα and/or Iκβ. HA-tagged IκBα or HA-tagged IκBβ cDNAs (Haskill et al., Cell 65:1281-1289, 1991) are translated in vitro in wheat germ extract in the presence of ³⁵S-methionine according to the manufacturer's instructions (Promega, Madison, Wis.). To phosphorylate IκBα or IκBβ, 1 μl of the extract containing the labeled protein is incubated for 90 minutes at 30° C. in a reaction mixture having a final volume of 30 μl: 100 μg HeLa or Jurkat cell extract (prepared as described by Alkalay et al., Proc. Natl. Acad. Sci. USA 92:10599, 1995), 2 mM ATP and 1 μM okadaic acid. Following incubation, 1 μl of anti-p65 serum is added, and the NF-κB immune complex is immobilized to Protein A-Sepharose® and subjected to in vitro ubiquitination in HeLa cell extract as described by Alkalay et al. Ubiquitinated proteins are separated by SDS-PAGE and visualized by autoradiography. The compositions are included in the ubiquitination reaction at different concentrations and tested for inhibition of pIκB specific ubiquitination. The inhibitory compositions are tested in a complementary ubiquitin-dependent in vitro degradation assay (Orian et al., J Biol. Chem. 270:21707, 1995; Stancovski et al., Mol. Cell. Biol. 15:7106, 1995).

### Example 7

### Maximum Tolerable Dose of the Compositions

A solution of Ganoderma lucidum, Salvia miltiorrhiza, and Scutellaria barbata representing 10× IC₅₀ was administered orally to SCID/nod mice. A solution of the extracted material (43.65 mg/ml.) was administered orally (1 ml/day/animal) to SCID/nod mice (25 gm; n=5) once a day for up to 14 days. The mice were monitored over a 28-day period for signs of stress following drug administration, including substantial loss of body weight, diarrhea, heavy panting, ruffling of hair, etc. On days 2 through 14, less than 13% body weight loss was observed (FIG. 6) and the animals were considered to be healthy. At the end of the period mice were terminated by CO₂ inhalation. Age-matched control mice (n=4) were treated with saline 1 ml/day for the 14 days. The data show that a daily dosage of 43.65 mg/ml/25 gm mouse of the extract is not toxic.

All publications and patent applications cited in this specification are herein incorporated by reference in their entirety as if each individual publication or patent application are specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for treating, preventing or inhibiting pain comprising administering to a subject a pharmaceutical composition comprising an effective amount of a two or more of an extract of Ganoderma lucidum, an extract of Salvia miltiorrhiza and an extract of Scutellaria barbata wherein each extract comprises about 1 to about 50 percent by weight of the composition.

2. The method according to claim 1, wherein the extract is a hot water extract.

3. The method according to claim 1, wherein the extract is an organic extract.

4. The method according to claim 1, wherein the extract is an ethyl acetate extract.

5. The method according to claim 1, wherein the composition displays at least one function selected from the group consisting of: anti-inflammation, anti-oxidation, inhibition of nociceptive pain, inhibition of chronic pain, inhibition of inflammatory pain, inhibition of tissue injury-induced pain, and inhibition of neuropathic pain.

6. The method according to claim 5, wherein the anti-inflammation function inhibits a cyclooxygenase (COX) activity.

7. The method according to claim 6, wherein the anti-inflammation function selectively inhibits COX-2 activity over COX-1 activity.

8. The method according to claim 5, wherein the anti-inflammation function inhibits an expression of a cyclooxygenase (COX) enzyme.

9. The method according to claim 8, wherein the anti-inflammation function selectively inhibits the expression COX-2 over COX-1.

10. The method according to claim 9, wherein the selective inhibition of the expression COX-2 over COX-1 is at least 2 fold.

11. The method according to claim 9, wherein the selective inhibition of the expression COX-2 over COX-1 is at least 5 fold.

12. The method according to claim 9, wherein the selective inhibition of the expression COX-2 over COX-1 is at least 20 fold.

13. The method according to claim 5, wherein the anti-inflammation function inhibits a nuclear accumulation of NF-κB protein.

14. The method according to claim 13, wherein the inhibition of a nuclear accumulation of NF-κB protein is at least 1.5 fold.

15. The method according to claim 13, wherein the inhibition of a nuclear accumulation of NF-κB protein is at least 2 fold.

16. The method according to claim 13, wherein the inhibition of a nuclear accumulation of NF-κB protein is mediated by an inhibition of degradation of IκB protein.

17. The method according to claim 16, wherein the inhibition of degradation of IκB protein is mediated by a reduction in ubiquitinylation of IκB protein.

18. The method according to claim 13, wherein the inhibition of a nuclear accumulation of NF-κB protein is accompanied by an inhibition of nitric oxide synthase.

19. The method according to claim 18, wherein the nitric oxide synthase is inducible nitric oxide synthase (iNOS)

20. The method according to claim 13, wherein the inhibition of a nuclear accumulation of NF-κB protein is in a cell subjected to proinflammatory cytokines.

21. The method according to claim 13, wherein the inhibition of a nuclear accumulation of NF-κB protein is in a cell subjected to interferon-gamma.

22. The method according to claim 13, wherein the inhibition of a nuclear accumulation of NF-κB protein is in a cell subjected to a lipopolysccharide (LPS).

23. The method according to claim 13, wherein the inhibition of a nuclear accumulation of NF-κB protein is caused by an inhibition of expression of NF-κB in response to interferon-gamma, lipopolysccharide (LPS), or proinflammatory cytokines.

24. The method according to claim 20 wherein the proinflammatory cytokine is TNF-alpha.

25. The method according to claim 1, further comprising an extract of Camellia sinensis (green tea).

26. The method according to claim 1, further comprising an extract of Hippophae rhamnoides.

27. The method according to claim 25, wherein the extract of Hippophae rhamnoides is an extract of H. rhamnoides leaf, H. rhamnoides berry or both.

28. The method according to claim 1 wherein the pain is a neuropathic pain caused by damage to the peripheral or central nervous system and maintained by aberrant somatosensory processing.

29. The method according to claim 28 wherein the composition inhibits an activity of a Group I mGluR.

30. The method according to claim 29 wherein the composition inhibits an activity of at least one of mGluR1 and mGluR5.

31. The method according to claim 28 wherein the composition inhibits an activity of a vanilloid receptor.

32. The method according to claim 1 wherein the pain is selected from the group consisting of acute pain, chronic pain, cancer pain, central pain, labor pain, myocardial infarction pain, pancreatic pain, colic pain, post-operative pain, headache pain, muscle pain, pain associated with intensive care, arthritic pain, neuropathic pain, and pain associated with a periodontal disease, including gingivitis and periodontitis.

33. The method according to claim 1 wherein the pain is an inflammatory pain selected from the group consisting of organ transplant rejection; reoxygenation injury resulting from organ transplantation, chronic inflammatory diseases of the joints, arthritis, rheumatoid arthritis, osteoarthritis, bone diseases associated with increased bone resorption, inflammatory lung diseases, asthma, adult respiratory distress syndrome, chronic obstructive airway disease, inflammatory diseases of the eye, corneal dystrophy, trachoma, onchocerciasis, uveitis, sympathetic ophthalmitis endophthalmitis, chronic inflammatory diseases of the gum, gingivitis, periodontitis, tuberculosis, leprosy, inflammatory diseases of the kidney, uremic complications, glomerulonephritis, nephrosis, inflammatory diseases of the skin, sclerodermatitis, psoriasis and eczema, inflammatory diseases of the central nervous system, chronic demyelinating diseases of the nervous system, multiple sclerosis, AIDS-related neurodegeneration, Alzheimer s disease, infectious meningitis, encephalomyelitis, Parkinson's disease, Huntington's disease, amyotrophic lateral sclerosis, viral or autoimmune encephalitis, autoimmune diseases, Type I and Type II diabetes mellitus, diabetic complications, diabetic cataract, glaucoma, retinopathy, nephropathy, microaluminuria, progressive diabetic nephropathy, polyneuropathy, mononeuropathies, autonomic neuropathy, gangrene of the feet, atherosclerotic coronary arterial disease, peripheral arterial disease, nonketotic hyperglycemic-hyperosmolar coma, foot ulcers, joint problems, skin or mucous membrane complication, immune-complex vasculitis, systemic lupus erythematosus (SLE), inflammatory diseases of the heart, cardiomyopathy, ischemic heart disease hypercholesterolemia, atherosclerosis, preeclampsia, chronic liver failure, brain and spinal cord trauma, and cancer.
